(19) United States
(12) Patent Application Publication
(10) Pub. No.: US 2024/0124599 A1
(43) Pub. Date: Apr. 18, 2024

(54) ANTI-BCMA HEAVY CHAIN-ONLY ANTIBODIES

(71) Applicant: TENEOBIO, INC., Thousand Oaks, CA (US)

(72) Inventors: Nathan Trinklein, Thousand Oaks, CA (US); Shelley Force Aldred, Thousand Oaks, CA (US); Katherine Harris, Thousand Oaks, CA (US); Wim van Schooten, Thousand Oaks, CA (US)

(21) Appl. No.: 18/468,406

(22) Filed: Sep. 15, 2023

Related U.S. Application Data
(63) Continuation of application No. 16/622,881, filed on Dec. 13, 2019.
(60) Provisional application No. 62/522,355, filed on Jun. 20, 2017.

(51) Int. Cl. C07K 16/28

(52) U.S. Cl. CPC C07K 16/2878; C07K 16/2809; C07K 2317/31; C07K 2317/51

(57) ABSTRACT

Anti-BCMA heavy chain-only antibodies (UniAb) and disclosed, along with methods of making such antibodies, compositions, including pharmaceutical compositions, comprising such antibodies, and their use to treat B-cell disorders characterized by the expression of BCMA.

### CROSS REFERENCE TO RELATED APPLICATIONS

This application is a Continuation of U.S. application Ser. No. 16/622,881, filed Dec. 13, 2019, which is a US National Stage entry under 35 USC 371, claiming priority to International Application No. PCT/US2018/038549, filed Jun. 20, 2018, which claims priority benefit of the filing date of U.S. Provisional Patent Application Ser. No. 62/522,355, filed on Jun. 20, 2017, the disclosures of which are herein incorporated by reference in their entireties.

### SEQUENCE LISTING

The instant application contains a Sequence Listing which has been submitted electronically in XML and is hereby incorporated by reference in its entirety. Said XML copy, created on Sep. 13, 2023, is named 60792.00021US02 (TNO-0004-C1-US); SL and is 34,000 bytes in size.

### FIELD OF THE INVENTION

The present invention concerns anti-BCMA heavy chain-only antibodies (UniAb). The invention further concerns methods of making such antibodies, compositions, including pharmaceutical compositions, comprising such antibodies, and their use to treat a B-cell disorder characterized by the expression of BCMA.

### BACKGROUND OF THE INVENTION

B-Cell Maturation Antigen (BCMA)

BCMA, also known as tumor necrosis factor superfamily member 17 (TNFRSF17) (UniProt Q02223), is a cell surface receptor exclusively expressed on plasma cells and plasmablasts. BCMA is a receptor for two ligands in the tumor necrosis factor (TNF) superfamily: APRIL (a proliferation-inducing ligand, also known as TNFSF13; TALL-2 and TRDL-1; the high affinity ligand for BCMA) and B cell activation factor (BAFF) (also known as BLyS; TALL-1; THANK; zTNF4; TNFSF20; and D8Ertd387e; the low affinity ligand for BCMA). APRIL and BAFF are growth factors that bind BCMA and promote survival of plasma cells. BCMA is also highly expressed on malignant plasma cells in human multiple myeloma (MM). Antibodies binding to BCMA are described, for example, in Gras et al., 1995, Int. Immunol. 7:1093-1106, WO200124811 and WO200124812. Anti-BCMA antibodies that cross-react with TACI are described in WO2002/066516. Bispecific antibodies against BCMA and CD3 are described, for example, in US 2013/0156769 A1 and US 2015/0376287 A1. An anti-BCMA antibody-MMAE or -MMAF conjugate has been reported to selectively induce killing of multiple myeloma cells (Tai et al., Blood 2014, 123(20): 3128-38). Ali et al., Blood 2016, 128(13):1688-700, have reported that in a clinical trial (#NCT02215967) chimeric antigen receptor (CAR) T cells targeting BCMA resulted in remission of multiple myeloma in human patients.

Heavy Chain-Only Antibodies

In a conventional IgG antibody, the association of the heavy chain and light chain is due in part to a hydrophobic interaction between the light chain constant region and the CH1 constant domain of the heavy chain. There are additional residues in the heavy chain framework 2 (FR2) and framework 4 (FR4) regions that also contribute to this hydrophobic interaction between the heavy and light chains.

It is known, however, that sera of camelids (sub-order Tylopoda which includes camels, dromedaries and llamas) contain a major type of antibodies composed solely of paired H-chains (heavy-chain only antibodies or UniAbs). The UniAbs of Camelidae (Camelus dromedarius, Camelus bactrianus, Lama glama, Lama guanaco, Lama alpaca and Lama vicugna) have a unique structure consisting of a single variable domain (VHH), a hinge region and two constant domains (CH2 and CH3), which are highly homologous to the CH2 and CH3 domains of classical antibodies. These UniAbs lack the first domain of the constant region (CH1) which is present in the genome, but is spliced out during mRNA processing. The absence of the CH1 domain explains the absence of the light chain in the UniAbs, since this domain is the anchoring place for the constant domain of the light chain. Such UniAbs naturally evolved to confer antigen-binding specificity and high affinity by three CDRs from conventional antibodies or fragments thereof (Muyldermans, 2001; J Biotechnol 74:277-302; Revets et al., 2005; Expert Opin Biol Ther 5:111-124). Cartilaginous fish, such as sharks, have also evolved a distinctive type of immunoglobulin, designated as IgNAR, which lacks the light polypeptide chains and is composed entirely by heavy chains. IgNAR molecules can be manipulated by molecular engineering to produce the variable domain of a single heavy chain polypeptide (vNARs) (Nuttall et al. Eur. J. Biochem. 270, 3543-3554 (2003); Nuttall et al. Function and Bioinformatics 55, 187-197 (2004); Dooley et al., Molecular Immunology 40, 25-33 (2003)).

The ability of heavy chain-only antibodies devoid of light chain to bind antigen was established in the 1960s (Jaton et al. (1968) Biochemistry, 7, 4185-4195). Heavy chain immunoglobulin physically separated from light chain retained 80% of antigen-binding activity relative to the tetrameric antibody. Sitia et al. (1990) Cell, 60, 781-790 demonstrated that removal of the CH1 domain from a rearranged mouse gene results in the production of a heavy chain-only antibody, devoid of light chain, in mammalian cell culture. The antibodies produced retained VH binding specificity and effector functions.

Heavy chain antibodies with a high specificity and affinity can be generated against a variety of antigens through immunization (van der Linden, R. H., et al. Biochim. Biophys. Acta. 1431, 37-46 (1999)) and the VHH portion can be readily cloned and expressed in yeast (Frenken, L. G. J., et al. J. Biotechnol. 78, 11-21 (2000)). Their levels of expression, solubility and stability are significantly higher than those of classical F(ab) or Fv fragments (Ghahroudi, M. A. et al. FEBS Lett. 414, 521-526 (1997)).

Mice in which the λ (lambda) light (L) chain locus and/or the λ and κ (kappa) L chain loci have been functionally silenced and antibodies produced by such mice are described in U.S. Pat. Nos. 7,541,513 and 8,367,888. Recombinant production of heavy chain-only antibodies in mice and rats has been reported, for example, in WO2006008548; U.S. Application Publication No. 20100122358; Nguyen et al., 2003, Immunology; 109(1), 93-101; Bruggemann et al., Crit. Rev. Immunol.; 2006, 26(5):377⁻⁹⁰; and Zou et al., 2007, J Exp Med; 204(13): 3271-3283. The production of knockout rats via embryo microinjections of zinc-finger nucleases is described in Geurts et al., 2009, Science, 325(5939):433. Soluble heavy chain-only antibodies and transgenic rodents comprising a heterologous heavy chain locus producing such antibodies are described in U.S. Pat. Nos. 8,883,150 and 9,365,655. CAR-T structures comprising single-domain antibodies as binding (targeting) domain are described, for example, in Iri-Sofia et al., 2011, Experimental Cell Research 317:2630-2641 and Jamnani et al., 2014, Biochim Biophys Acta, 1840:378-386.

### SUMMARY OF THE INVENTION

The present invention concerns heavy chain-only antibodies binding to human B-Cell Maturation Antigen (BCMA).

In one aspect, the invention concerns heavy chain-only anti-BCMA antibodies comprising a heavy chain variable region comprising:
(a) a CDR1 having two or fewer substitutions in any of the amino acid sequences of SEQ ID NO:1, 2, or 3; and/or
(b) a CDR2 having two or fewer substitutions in any of the amino acid sequences of SEQ ID NOs: 4 to 7; and/or
(c) a CDR3 having two or fewer substitutions in any of the amino acid sequences of SEQ ID NOs:8 to 11.

In one embodiment, the CDR1, CDR2, and CDR3 sequences are present in a human framework.

In another embodiment, the heavy chain-only anti-BCMA antibodies further comprise a heavy chain constant region sequence in the absence of a CH1 sequence.

In yet another embodiment, the heavy chain-only anti-BCMA antibody comprises:
(a) a CDR1 sequence selected from the group consisting of SEQ ID NOs: 1 to 3; and/or
(b) a CDR2 sequence selected from the group consisting of SEQ ID NOs: 4 to 7; and/or
(c) a CDR3 sequence selected from the group consisting of SEQ ID NOs: 8 to 11.

In a further embodiment, the heavy chain-only antibody comprises:
(a) a CDR1 sequence selected from the group consisting of SEQ ID NOs: 1 to 3; and
(b) a CDR2 sequence selected from the group consisting of SEQ ID NOs: 4 to 7; and
(c) a CDR3 sequence selected from the group consisting of SEQ ID NOs:8 to 11.

In a still further embodiment, the heavy chain only antibody comprises
(i) a CDR1 sequence of SEQ ID NO: 1, a CDR2 sequence of SEQ ID NO: 4, and a CDR3 sequence of SEQ ID NO: 8; or
(ii) a CDR1 sequence of SEQ ID NO: 2, a CDR2 sequence of SEQ ID NO: 5, and a CDR3 sequence of SEQ ID NO: 9; or
(iii) a CDR1 sequence of SEQ ID NO: 2, a CDR2 sequence of SEQ ID NO: 6, and a CDR3 sequence of SEQ ID NO: 10; or
(iv) a CDR1 sequence of SEQ ID NO: 3, a CDR2 sequence of SEQ ID NO: 7, and a CDR3 sequence of SEQ ID NO: 11.

In another embodiment, the heavy chain-only anti-BCMA antibody comprises a heavy chain variable region having at least 95% sequence identity to any of the sequences of SEQ ID NOs: 12 to 15.

In a further embodiment, the heavy chain-only anti-BCMA antibody comprises a heavy chain variable region sequence selected from the group consisting of SEQ ID NOs: 12 to 15.

In a further aspect, the invention concerns a heavy chain-only anti-BCMA antibody comprising a heavy chain variable region comprising a heavy chain variable comprising
(a) a CDR1 sequence of the formula

G F T F X1 X2 X3 A (SEQ ID NO: 20)
where
   X1 is S or T;
   X2 is S or N;
   X3 is H or Y, or
   (b) a CDR2 sequence of the formula

I S G X4 G X5 X6 X7 (SEQ ID NO: 21)
where
   X4 is S or N;
   X5 is D or R;
   X6 is T, F or Y; or
   X7 is T or I,
(c) a CDR3 sequence selected from the group consisting of AKDGGETLVDS (SEQ ID NO: 8), AKDEDGGSLLGY (SEQ ID NO: 9), AKDEDGGSLLGH (SEQ ID NO: 10), and AKEGTGANSSLADY (SEQ ID NO: 11).

In another aspect, the invention concerns a heavy chain-only antibody binding to human B-Cell Maturation Antigen (BCMA) comprising a heavy chain variable region comprising CDR1, CDR2 and CDR3 sequences in a human VH framework wherein the CDR sequences have 2 or fewer amino acid substitutions in a CDR sequence selected from the group consisting of SEQ ID NOs:1-11.

In one embodiment, the anti-BCMA heavy chain-only antibody comprises a heavy chain variable region comprising CDR1, CDR2 and CDR3 sequences in a human VH framework wherein the CDR sequences are selected from the group consisting of SEQ ID NOs:1-11.

In another embodiment, the invention concerns an anti-BCMA heavy chain-only antibody comprising a heavy chain variable region comprising
(i) a CDR1 sequence of SEQ ID NO: 1, a CDR2 sequence of SEQ ID NO: 4, and a CDR3 sequence of SEQ ID NO: 8; or
(ii) a CDR1 sequence of SEQ ID NO: 2, a CDR2 sequence of SEQ ID NO: 5, and a CDR3 sequence of SEQ ID NO: 9; or
(iii) a CDR1 sequence of SEQ ID NO: 2, a CDR2 sequence of SEQ ID NO: 6, and a CDR3 sequence of SEQ ID NO: 10; or
(iv) a CDR1 sequence of SEQ ID NO: 3, a CDR2 sequence of SEQ ID NO: 7, and a CDR3 sequence of SEQ ID NO: 11,
in a human VH framework.

In all aspects and embodiments, the heavy chain-only antibodies may be multi-specific, such as bispecific, and may, for example, bind to two different BCMA proteins or two different epitopes on the same BCMA protein.

In one embodiment, the heavy chain-only antibody has binding affinity to an effector cell.

In a second embodiment, the heavy chain-only antibody has binding affinity to a T-cell antigen, such as CD3.

In a third embodiment, the heavy chain-only antibody is in a CAR-T format.

In another aspect, the invention concerns a pharmaceutical composition comprising a heavy chain-only antibody as hereinabove described.

In yet another aspect, the invention concerns a method for the treatment of a B-cell disorder characterized by the expression of BCMA, the method comprising administering to a subject with said disorder a heavy chain-only antibody, or a pharmaceutical composition, as hereinabove described.

In one embodiment, the B-cell disorder is multiple myeloma (MM).

In another embodiment, the B-cell disorder is systemic lupus erythematosus (SLE).

In a further aspect, the invention concerns a polynucleotide encoding an anti-BCMA heavy chain-only antibody as described herein.

In a still further aspect, the invention concerns a vector comprising a polynucleotide encoding an anti-BCMA heavy chain-only antibody as described herein.

In another aspect, the invention concerns a cell comprising a polynucleotide encoding an anti-BCMA heavy chain-only antibody as described herein, or a vector comprising such polynucleotide.

In yet another aspect, the invention concerns a method of producing an anti-BCMA heavy chain-only antibody as described herein, the method comprising growing a cell comprising a polynucleotide encoding an anti-BCMA heavy chain-only antibody as described herein, or a vector comprising such polynucleotide, under conditions permissive for expression of the protein, and isolating the antibody from the cell and/or the cell culture medium.

In a further aspect, the invention concerns a method of making an anti-BCMA heavy chain-only antibody as described herein, the method comprising immunizing a UniRat animal with BCMA and identifying BCMA-binding heavy chain sequences.

These and further aspect will be further explained in the rest of the disclosure, including the Examples.

### BRIEF DESCRIPTION OF THE DRAWINGS

FIG. 1 shows the CDR1, CDR2 and CDR3 amino acid sequences of 4 heavy chain-only anti-BCMA antibodies of the invention.

FIG. 2 shows the heavy chain variable region amino acid sequences of 4 heavy chain-only anti-BCMA antibodies of the invention.

FIG. 3 shows the nucleic acid sequence encoding the heavy chain variable region sequences of 4 heavy chain-only anti-BCMA antibodies of the invention.

FIG. 4 shows binding to BCMA protein and BCMA-expressing cell lines of 4 heavy-chain antibodies. Column 1 indicates the clone ID of the HCAb. Column 2 indicates the family ID of the HCAb based on the CDR3 sequence. Column 3 indicates the CDR1 amino acid sequence (SEQ ID NOS 1-2 and 2-3, respectively, in order of appearance). Column 4 indicates the CDR2 amino acid sequence (SEQ ID NOS 4-7, respectively, in order of appearance). Column 5 indicates the CDR3 amino acid sequence (SEQ ID NOS 8-11, respectively, in order of appearance). Column 6 indicates the concentration of the expressed HCAb in ug/mL. Column 7 indicates the mean fluorescent intensity of cell binding to H929 human cells that express BCMA. Column 8 indicates the mean fluorescent intensity of cell binding to CHO cells that express cyno BCMA. Column 9 indicates the ELISA fold over background signal of human BCMA protein binding. Column 10 indicates the ELISA fold over background signal of cyno BCMA protein binding. Column 11 indicates the ELISA fold over background signal of lambda protein binding, an off-target control. Column 12 indicates the ELISA fold over background signal of a multi-tag protein binding, an off-target control. Column 13 indicates the binding off-rate to human BCMA protein measured by the Octet. Column 14 indicates the binding off-rate to cyno BCMA protein measured by the Octet.

FIG. 5 is a graphic illustration of an scFv CAR-T construct, a monospecific human VH CAR-T construct, and a bispecific human VH CAR-T construct.

### DETAILED DESCRIPTION OF THE PREFERRED EMBODIMENTS

The practice of the present invention will employ, unless otherwise indicated, conventional techniques of molecular biology (including recombinant techniques), microbiology, cell biology, biochemistry, and immunology, which are within the skill of the art. Such techniques are explained fully in the literature, such as, “Molecular Cloning: A Laboratory Manual”, second edition (Sambrook et al., 1989); “Oligonucleotide Synthesis” (M. J. Gait, ed., 1984); “Animal Cell Culture” (R. I. Freshney, ed., 1987); “Methods in Enzymology” (Academic Press, Inc.); “Current Protocols in Molecular Biology” (F. M. Ausubel et al., eds., 1987, and periodic updates); “PCR: The Polymerase Chain Reaction”, (Mullis et al., ed., 1994); “A Practical Guide to Molecular Cloning” (Perbal Bernard V., 1988); “Phage Display: A Laboratory Manual” (Barbas et al., 2001); Harlow, Lane and Harlow, Using Antibodies: A Laboratory Manual: Portable Protocol No. I, Cold Spring Harbor Laboratory (1998); and Harlow and Lane, Antibodies: A Laboratory Manual, Cold Spring Harbor Laboratory; (1988).

Where a range of values is provided, it is understood that each intervening value, to the tenth of the unit of the lower limit unless the context clearly dictates otherwise, between the upper and lower limit of that range and any other stated or intervening value in that stated range is encompassed within the invention. The upper and lower limits of these smaller ranges may independently be included in the smaller ranges is also encompassed within the invention, subject to any specifically excluded limit in the stated range. Where the stated range includes one or both of the limits, ranges excluding either or both of those included limits are also included in the invention.

Unless indicated otherwise, antibody residues herein are numbered according to the Kabat numbering system (e.g., Kabat et al., Sequences of Immunological Interest. 5th Ed. Public Health Service, National Institutes of Health, Bethesda, Md. (1991)).

In the following description, numerous specific details are set forth to provide a more thorough understanding of the present invention. However, it will be apparent to one of skill in the art that the present invention may be practiced without one or more of these specific details. In other instances, well-known features and procedures well known to those skilled in the art have not been described in order to avoid obscuring the invention.

All references cited throughout the disclosure, including patent applications and publications, are incorporated by reference herein in their entirety.

### I. Definitions

By “comprising” it is meant that the recited elements are required in the composition/method/kit, but other elements may be included to form the composition/method/kit etc. within the scope of the claim.

By “consisting essentially of”, it is meant a limitation of the scope of composition or method described to the specified materials or steps that do not materially affect the basic and novel characteristic(s) of the subject invention.

By “consisting of”, it is meant the exclusion from the composition, method, or kit of any element, step, or ingredient not specified in the claim.

The term “monoclonal antibody” as used herein refers to an antibody obtained from a population of substantially homogeneous antibodies, i.e., the individual antibodies comprising the population are identical except for possible naturally occurring mutations that may be present in minor amounts. Monoclonal antibodies are highly specific, being directed against a single antigenic site. Furthermore, in contrast to conventional (polyclonal) antibody preparations which typically include different antibodies directed against different determinants (epitopes), each monoclonal antibody is directed against a single determinant on the antigen.

The terms “heavy chain-only antibody,” “heavy-chain antibody” and “UniAb” are used interchangeably, and refer, in the broadest sense, to antibodies lacking the light chain of a conventional antibody. Since the homodimeric UniAbs lack a light chain and thus a VL domain, the antigen is recognized by one single domain, i.e., the variable domain of the heavy chain of a heavy-chain antibody (VH). The term specifically includes, without limitation, homodimeric antibodies comprising the VH antigen-binding domain and the CH2 and CH3 constant domains, in the absence of the CH1 domain; functional (antigen-binding) variants of such antibodies, soluble VH variants, IgNAR comprising a homodimer of one variable domain (V-NAR) and five C-like constant domains (C-NAR) and functional fragments thereof; and soluble single domain antibodies (sUniDabs). In one embodiment, the heavy chain-only antibody is composed of the variable region antigen-binding domain composed of framework 1, CDR1, framework 2, CDR2, framework 3, CDR3, and framework 4. In one embodiment, the heavy chain-only antibody is composed of an antigen-binding domain, at least part of a hinge region and CH2 and CH3 domains. In another embodiment, the heavy chain-only antibody is composed of an antigen-binding domain, at least part of a hinge region and a CH2 domain. In a further embodiment, the heavy chain-only antibody is composed of an antigen-binding domain, at least part of a hinge region and a CH3 domain. Heavy chain-only antibodies in which the CH2 and/or CH3 domain is truncated are also included herein. In a further embodiment the heavy chain is composed of an antigen binding domain, and at least one CH (CH1, CH2, CH3, or CH4) domain but no hinge region. The heavy chain-only antibody can be in the form of a dimer, in which two heavy chains are disulfide bonded other otherwise, covalently or non-covalently attached with each other. The heavy chain-only antibody may belong to the IgG subclass, but antibodies belonging to other subclasses, such as IgM, IgA, IgD and IgE subclass, are also included herein. In a particular embodiment, the heavy chain antibody is of the IgG1, IgG2, IgG3, or IgG4 subtype, in particular IgG1 subtype. In one embodiment, the heavy chain-only antibodies herein are used as a binding (targeting) domain of a chimeric antigen receptor (CAR).

The term “BCMA” as used herein relates to human B cell maturation antigen, also known as BCMA, CD269, and TNFRSF17 (UniProt Q02223), which is a member of the tumor necrosis receptor superfamily that is preferentially expressed in differentiated plasma cells. The extracellular domain of human BCMA consists, according to UniProt of amino acids 1-54 (or 5-51).

The terms “anti-BCMA heavy chain-only antibody” and “BCMA heavy chain-only antibody” are used herein to refer to a heavy chain-only antibody as hereinabove defined, immunospecifically binding to BCMA.

The term “variable”, as used in connection with antibodies, refers to the fact that certain portions of the antibody variable domains differ extensively in sequence among antibodies and are used in the binding and specificity of each particular antibody for its particular antigen. However, the variability is not evenly distributed throughout the variable domains of antibodies. It is concentrated in three segments called hypervariable regions both in the light chain and the heavy chain variable domains. The more highly conserved portions of variable domains are called the framework regions (FRs). The variable domains of native heavy and light chains each comprise four FRs, largely adopting a $-sheet configuration, connected by three hypervariable regions, which form loops connecting, and in some cases forming part of, the $-sheet structure. The hypervariable regions in each chain are held together in close proximity by the FRs and, with the hypervariable regions from the other chain, contribute to the formation of the antigen-binding site of antibodies (see Kabat et al., Sequences of Proteins of Immunological Interest, 5th Ed. Public Health Service, National Institutes of Health, Bethesda, MD. (1991)). The constant domains are not involved directly in binding an antibody to an antigen, but exhibit various effector functions, such as participation of the antibody in antibody dependent cellular cytotoxicity (ADCC).

The term “hypervariable region” when used herein refers to the amino acid residues of an antibody which are responsible for antigen-binding. The hypervariable region generally comprises amino acid residues from a “complementarity determining region” or “CDR” (e.g., residues 31-35 (H1), 50-65 (H2) and 95-102 (H3) in the heavy chain variable domain; Kabat et al., Sequences of Proteins of Immunological Interest, 5th Ed. Public Health Service, National Institutes of Health, Bethesda, MD. (1991)) and/or those residues from a “hypervariable loop” residues 26-32 (H1), 53-55 (H2) and 96-101 (H3) in the heavy chain variable domain; Chothia and Lesk J. Mol. Biol. 196:901-917 (1987)). “Framework Region” or “FR” residues are those variable domain residues other than the hypervariable region residues as herein defined.

Exemplary CDR designations are shown herein, however one of skill in the art will understand that a number of definitions of the CDRs are commonly in use, including the Kabat definition (see “Zhao et al. A germline knowledge based computational approach for determining antibody complementarity determining regions.” Mol Immunol. 2010; 47:694-700), which is based on sequence variability and is the most commonly used. The Chothia definition is based on the location of the structural loop regions (Chothia et al. “Conformations of immunoglobulin hypervariable regions.” Nature. 1989; 342:877⁻⁸⁸³). Alternative CDR definitions of interest include, without limitation, those disclosed by Honegger, “Yet another numbering scheme for immunoglobulin variable domains: an automatic modeling and analysis tool.” J Mol Biol. 2001; 309:657-670; Ofran et al. “Automated identification of complementarity determining regions (CDRs) reveals peculiar characteristics of CDRs and B cell epitopes.” J Immunol. 2008; 181:6230-6235; Almagro “Identification of differences in the specificity-determining residues of antibodies that recognize antigens of different size: implications for the rational design of antibody repertoires.” J Mol Recognit. 2004; 17:132-143; and Padlan et al. “Identification of specificity-determining residues in antibodies.” Faseb J. 1995; 9:133-139., each of which is herein specifically incorporated by reference.

The term “2 (two) or fewer substitutions” in an amino acid sequence is used herein to mean 2 (two), 1 (one) or 0 (zero) substitutions in the reference amino acid sequence.

“Percent (%) amino acid sequence identity” with respect to a reference polypeptide sequence is defined as the percentage of amino acid residues in a candidate sequence that are identical with the amino acid residues in the reference polypeptide sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as BLAST, BLAST-2, ALIGN or Megalign (DNASTAR) software. Those skilled in the art can determine appropriate parameters for aligning sequences, including any algorithms needed to achieve maximal alignment over the full length of the sequences being compared. For purposes herein, however, % amino acid sequence identity values are generated using the sequence comparison computer program ALIGN-2.

An “isolated” antibody is one which has been identified and separated and/or recovered from a component of its natural environment. Contaminant components of its natural environment are materials which would interfere with diagnostic or therapeutic uses for the antibody, and may include enzymes, hormones, and other proteinaceous or nonproteinaceous solutes. In preferred embodiments, the antibody will be purified (1) to greater than 95% by weight of antibody as determined by the Lowry method, and most preferably more than 99% by weight, (2) to a degree sufficient to obtain at least 15 residues of N-terminal or internal amino acid sequence by use of a spinning cup sequenator, or (3) to homogeneity by SDS-PAGE under reducing or nonreducing conditions using Coomassie blue or, preferably, silver stain. Isolated antibody includes the antibody in situ within recombinant cells since at least one component of the antibody's natural environment will not be present. Ordinarily, however, isolated antibody will be prepared by at least one purification step.

Antibodies of the invention include multi-specific antibodies. Multi-specific antibodies have more than one binding specificity. The term “multi-specific” specifically includes “bispecific” and “trispecific,” as well as higher-order independent specific binding affinities, such as higher-order polyepitopic specificity, as well as tetravalent antibodies and antibody fragments. “Multi-specific” antibodies specifically include antibodies comprising a combination of different binding entities as well as antibodies comprising more than one of the same binding entity. The terms “multi-specific antibody,” multi-specific single chain-only antibody” and “multi-specific UniAb” are used herein in the broadest sense and cover all antibodies with more than one binding specificity.

The term “valent” as used herein refers to a specified number of binding sites in an antibody molecule.

A “multi-valent” antibody has two or more binding sites. Thus, the terms “bivalent”, “trivalent”, and “tetravalent” refers to the presence of two binding sites, three binding sites, and four binding sites, respectively. Thus, a bispecific antibody according to the invention is at least bivalent and may be trivalent, tetravalent, or otherwise multi-valent.

A large variety of methods and protein configurations are known and used for the preparation of bispecific monoclonal antibodies (BsMAB), tri-specific antibodies, and the like.

The term “bispecific three-chain antibody like molecule” or “TCA” is used herein to refer to antibody-like molecules comprising, consisting essentially of, or consisting of three polypeptide subunits, two of which comprise, consist essentially of, or consist of one heavy and one light chain of a monoclonal antibody, or functional antigen-binding fragments of such antibody chains, comprising an antigen-binding region and at least one CH domain. This heavy chain/light chain pair has binding specificity for a first antigen. The third polypeptide subunit comprises, consists essentially of, or consists of a heavy chain only antibody comprising an Fc portion comprising CH2 and/or CH3 and/or CH4 domains, in the absence of a CH1 domain, and an antigen binding domain that binds an epitope of a second antigen or a different epitope of the first antigen, where such binding domain is derived from or has sequence identity with the variable region of an antibody heavy or light chain. Parts of such variable region may be encoded by VH and/or VL gene segments, D and JH gene segments, or JL gene segments. The variable region may be encoded by rearranged VHDJH, VLDJH, VHJL, or VLJL gene segments. A TCA protein makes use of a heavy chain-only antibody as hereinabove defined.

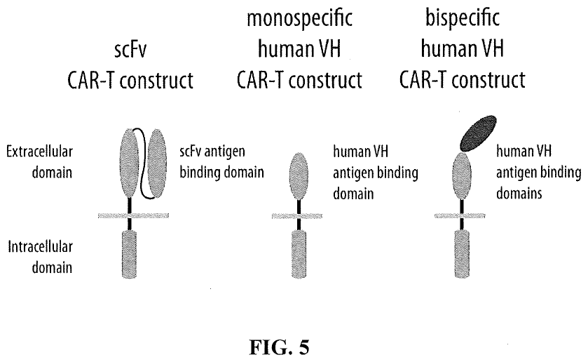

The term “chimeric antigen receptor” or “CAR” is used herein in the broadest sense to refer to an engineered receptor, which grafts a desired binding specificity (e.g., the antigen-binding region of a monoclonal antibody or other ligand) to membrane-spanning and intracellular-signaling domains. Typically, the receptor is used to graft the specificity of a monoclonal antibody onto a T cell to create a chimeric antigen receptors (CAR). (J Natl Cancer Inst, 2015; 108(7):dvj439; and Jackson et al., Nature Reviews Clinical Oncology, 2016; 13:370-383.) Representative monospecific and bispecific CAR-T constructs comprising a human VH extracellular binding domain are shown in FIG. 5, in comparison to an scFv CAR-T construct.

By “human idiotype” is meant a polypeptide sequence epitope present on a human antibody in the immunoglobulin heavy and/or light chain variable region. The term “human idiotype” as used herein includes both naturally occurring sequences of a human antibody, as well as synthetic sequences substantially identical to the polypeptide found in naturally occurring human antibodies. By “substantially” is meant that the degree of amino acid sequence identity is at least about 85%-95%. Preferably, the degree of amino acid sequence identity is greater than 90%, more preferably greater than 95%.

By a “chimeric antibody” or a “chimeric immunoglobulin” is meant an immunoglobulin molecule comprising amino acid sequences from at least two different Ig loci, e.g., a transgenic antibody comprising a portion encoded by a human Ig locus and a portion encoded by a rat Ig locus. Chimeric antibodies include transgenic antibodies with non-human Fc-regions or artificial Fc-regions, and human idiotypes. Such immunoglobulins can be isolated from animals of the invention that have been engineered to produce such chimeric antibodies.

Antibody “effector functions” refer to those biological activities attributable to the Fc region (a native sequence Fc region or amino acid sequence variant Fc region) of an antibody. Examples of antibody effector functions include Clq binding; complement dependent cytotoxicity; Fc receptor binding; antibody-dependent cell-mediated cytotoxicity (ADCC); phagocytosis; down regulation of cell surface receptors (e.g., B cell receptor; BCR), etc.

“Antibody-dependent cell-mediated cytotoxicity” and “ADCC” refer to a cell-mediated reaction in which nonspecific cytotoxic cells that express Fc receptors (FcRs) (e.g., Natural Killer (NK) cells, neutrophils, and macrophages) recognize bound antibody on a target cell and subsequently cause lysis of the target cell. The primary cells for mediating ADCC, NK cells, express FcγRIII only, whereas monocytes express FcγRI, FcγRII and FcγRIII. FcR expression on hematopoietic cells in summarized is Table 3 on page 464 of Ravetch and Kinet, Annu. Rev. Immunol 9:457⁻⁹² (1991). To assess ADCC activity of a molecule of interest, an in vitro ADCC assay, such as that described in U.S. Pat. No. 5,500,362 or 5,821,337 may be performed. Useful effector cells for such assays include peripheral blood mononuclear cells (PBMC) and Natural Killer (NK) cells. Alternatively, or additionally, ADCC activity of the molecule of interest may be assessed in vivo, e.g., in an animal model such as that disclosed in Clynes et al. PNAS (USA) 95:652-656 (1998).

“Human effector cells” are leukocytes which express one or more FcRs and perform effector functions. Preferably, the cells express at least FcγRIII and perform ADCC effector function. Examples of human leukocytes which mediate ADCC include peripheral blood mononuclear cells (PBMC), natural killer (NK) cells, monocytes, cytotoxic T cells and neutrophils; with PBMCs and NK cells being preferred. The effector cells may be isolated from a native source thereof, e.g., from blood or PBMCs as described herein.

“Complement dependent cytotoxicity” or “CDC” refers to the ability of a molecule to lyse a target in the presence of complement. The complement activation pathway is initiated by the binding of the first component of the complement system (Clq) to a molecule (e.g., an antibody) complexed with a cognate antigen. To assess complement activation, a CDC assay, e.g., as described in Gazzano-Santoro et al., J. Immunol. Methods 202:163 (1996), may be performed.

“Binding affinity” refers to the strength of the sum total of noncovalent interactions between a single binding site of a molecule (e.g., an antibody) and its binding partner (e.g., an antigen). Unless indicated otherwise, as used herein, “binding affinity” refers to intrinsic binding affinity which reflects a 1:1 interaction between members of a binding pair (e.g., antibody and antigen). The affinity of a molecule X for its partner Y can generally be represented by the dissociation constant (Kd). Affinity can be measured by common methods known in the art. Low-affinity antibodies generally bind antigen slowly and tend to dissociate readily, whereas high-affinity antibodies generally bind antigen faster and tend to remain bound.

As used herein, the “Kd” or “Kd value” refers to a dissociation constant determined by BioLayer Interferometry, using an Octet QK384 instrument (Fortebio Inc., Menlo Park, CA) in kinetics mode. For example, anti-mouse Fc sensors are loaded with mouse-Fc fused antigen and then dipped into antibody-containing wells to measure concentration dependent association rates (kon). Antibody dissociation rates (koff) are measured in the final step, where the sensors are dipped into wells containing buffer only. The Kd is the ratio of koff/kon. (For further details see, Concepcion, J, et al., Comb Chem High Throughput Screen, 12(8), 791-800, 2009).

An “epitope” is the site on the surface of an antigen molecule to which a single antibody molecule binds. Generally an antigen has several or many different epitopes and reacts with many different antibodies. The term specifically includes linear epitopes and conformational epitopes.

“Epitope mapping” is the process of identifying the binding sites, or epitopes, of antibodies on their target antigens. Antibody epitopes may be linear epitopes or conformational epitopes. Linear epitopes are formed by a continuous sequence of amino acids in a protein. Conformational epitopes are formed of amino acids that are discontinuous in the protein sequence, but which are brought together upon folding of the protein into its three-dimensional structure.

“Polyepitopic specificity” refers to the ability to specifically bind to two or more different epitopes on the same or different target(s).

An antibody binds “essentially the same epitope” as a reference antibody, when the two antibodies recognize identical or sterically overlapping epitopes. The most widely used and rapid methods for determining whether two epitopes bind to identical or sterically overlapping epitopes are competition assays, which can be configured in all number of different formats, using either labeled antigen or labeled antibody. Usually, the antigen is immobilized on a 96-well plate, and the ability of unlabeled antibodies to block the binding of labeled antibodies is measured using radioactive or enzyme labels.

The terms “treatment”, “treating” and the like are used herein to generally mean obtaining a desired pharmacologic and/or physiologic effect. The effect may be prophylactic in terms of completely or partially preventing a disease or symptom thereof and/or may be therapeutic in terms of a partial or complete cure for a disease and/or adverse effect attributable to the disease. “Treatment” as used herein covers any treatment of a disease in a mammal, and includes: (a) preventing the disease from occurring in a subject which may be predisposed to the disease but has not yet been diagnosed as having it; (b) inhibiting the disease, i.e., arresting its development; or (c) relieving the disease, i.e., causing regression of the disease. The therapeutic agent may be administered before, during or after the onset of disease or injury. The treatment of ongoing disease, where the treatment stabilizes or reduces the undesirable clinical symptoms of the patient, is of particular interest. Such treatment is desirably performed prior to complete loss of function in the affected tissues. The subject therapy may be administered during the symptomatic stage of the disease, and in some cases after the symptomatic stage of the disease.

A “therapeutically effective amount” is intended for an amount of active agent which is necessary to impart therapeutic benefit to a subject. For example, a “therapeutically effective amount” is an amount which induces, ameliorates or otherwise causes an improvement in the pathological symptoms, disease progression or physiological conditions associated with a disease or which improves resistance to a disorder.

The terms “subject,” “individual,” and “patient” are used interchangeably herein to refer to a mammal being assessed for treatment and/or being treated. In an embodiment, the mammal is a human. The terms “subject,” “individual,” and “patient” encompass, without limitation, individuals having cancer, individuals with autoimmune diseases, with pathogen infections, and the like. Subjects may be human, but also include other mammals, particularly those mammals useful as laboratory models for human disease, e.g., mouse, rat, etc.

The term “CD3” refers to the human CD3 protein multi-subunit complex. The CD3 protein multi-subunit complex is composed to 6 distinctive polypeptide chains. These include a CD3γ chain (SwissProt P09693), a CD36 chain (SwissProtPO4234), two CD3e chains (SwissProt P07766), and one CD3ζ chain homodimer (SwissProt 20963), and which is associated with the T cell receptor a and β chain. The term “CD3” includes any CD3 variant, isoform and species homolog which is naturally expressed by cells (including T cells) or can be expressed on cells transfected with genes or cDNA encoding those polypeptides, unless noted.

A “BCMA×CD3 antibody” is a multispecific heavy chain-only antibody, such as a bispecific heavy chain-only antibody, which comprises two different antigen-binding regions, one of which binds specifically to the antigen BCMA and one of which binds specifically to CD3.

The term “pharmaceutical formulation” refers to a preparation which is in such form as to permit the biological activity of the active ingredient to be effective, and which contains no additional components which are unacceptably toxic to a subject to which the formulation would be administered. Such formulations are sterile. “Pharmaceutically acceptable” excipients (vehicles, additives) are those which can reasonably be administered to a subject mammal to provide an effective dose of the active ingredient employed.

A “sterile” formulation is aseptic or free or essentially free from all living microorganisms and their spores. A “frozen” formulation is one at a temperature below 0° C.

A “stable” formulation is one in which the protein therein essentially retains its physical stability and/or chemical stability and/or biological activity upon storage. Preferably, the formulation essentially retains its physical and chemical stability, as well as its biological activity upon storage. The storage period is generally selected based on the intended shelf-life of the formulation. Various analytical techniques for measuring protein stability are available in the art and are reviewed in Peptide and Protein Drug Delivery, 247-301. Vincent Lee Ed., Marcel Dekker, Inc., New York, N.Y., Pubs. (1991) and Jones. A. Adv. Drug Delivery Rev. 10: 29⁻⁹⁰) (1993), for example. Stability can be measured at a selected temperature for a selected time period. Stability can be evaluated qualitatively and/or quantitatively in a variety of different ways, including evaluation of aggregate formation (for example using size exclusion chromatography, by measuring turbidity, and/or by visual inspection); by assessing charge heterogeneity using cation exchange chromatography, image capillary isoelectric focusing (icIEF) or capillary zone electrophoresis; amino-terminal or carboxy-terminal sequence analysis; mass spectrometric analysis; SDS-PAGE analysis to compare reduced and intact antibody; peptide map (for example tryptic or LYS-C) analysis; evaluating biological activity or antigen binding function of the antibody; etc. Instability may involve any one or more of: aggregation, deamidation (e.g., Asn deamidation), oxidation (e.g., Met oxidation), isomerization (e.g., Asp isomeriation), clipping/hydrolysis/fragmentation (e.g., hinge region fragmentation), succinimide formation, unpaired cysteine(s), N-terminal extension, C-terminal processing, glycosylation differences, etc.

### II. Detailed Description

### Anti-BCMA Antibodies

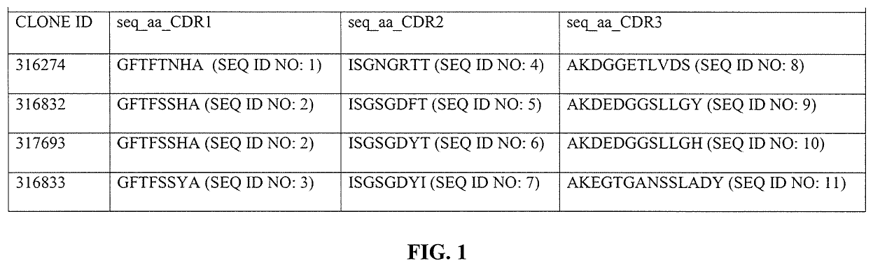

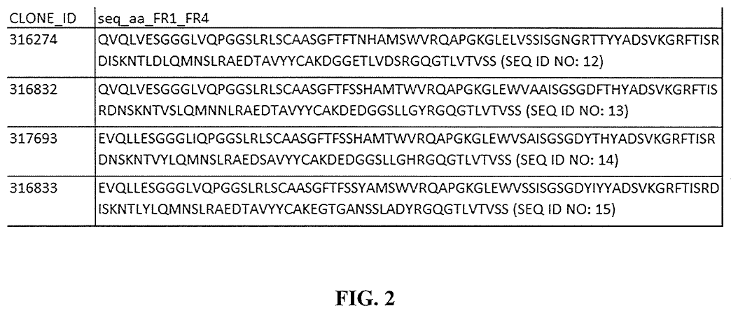

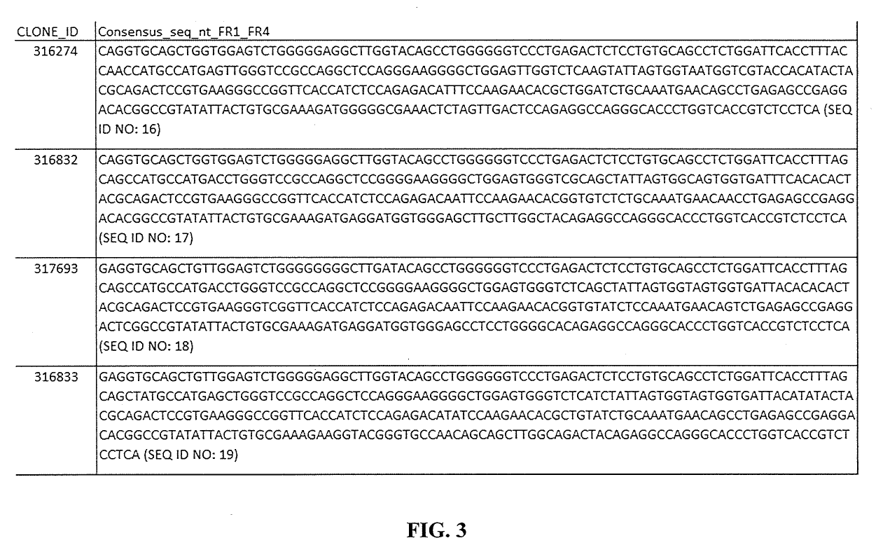

The present invention provides heavy chain-only antibodies (UniAbs) that bind to human BCMA. The anti-BCMA UniAbs of the invention comprise a set of CDR sequences as defined herein and shown in FIG. 1, and are exemplified by the provided heavy chain variable region (VH) sequences of SEQ ID NOs 12 to 15, set forth in FIG. 2, encoded by the nucleic acid sequences of SEQ ID NOs: 16-19, set forth in FIG. 3. These antibodies provide a number of benefits that contribute to utility as clinically therapeutic agent(s). The antibodies include members with a range of binding affinities, allowing the selection of a specific sequence with a desired binding affinity.

A suitable antibody may be selected from those provided herein for development and therapeutic or other use, including, without limitation, use as a bispecific or tri-specific antibody, or part of a CAR-T structure, e.g., as shown in FIG. 5.

Determination of affinity for a candidate protein can be performed using methods known in the art, such as Biacore measurements. Members of the antibody family may have an affinity for BCMA with a Kd of from about 10⁻⁶ to around about 10⁻¹, including without limitation: from about 10⁻⁶ to around about 10⁻¹⁰; from about 10⁻⁶ to around about 10⁻⁹; from about 10⁻⁶ to around about 10⁻⁸; from about 10⁻⁸ to around about 10⁻¹; from about 10⁻⁸ to around about 10⁻¹⁰; from about 10⁻⁸ to around about 10⁻⁹; from about 10⁻⁹ to around about 10⁻¹; from about 10⁻⁹ to around about 10⁻¹⁰; or any value within these ranges. The affinity selection may be confirmed with a biological assessment for modulating, e.g., blocking, a BCMA biological activity, including in vitro assays, pre-clinical models, and clinical trials, as well as assessment of potential toxicity.

Members of the antibody family herein are not cross-reactive with the BCMA protein of Cynomolgus macaque, but can be engineered to provide cross-reactivity with the BCMA protein of Cynomolgus macaque, or with the BCMA of any other animal species, if desired.

In some embodiments, the anti-BCMA UniAb antibodies herein comprise a VH domain, comprising CDR1, CDR2 and CDR3 sequences in a human VH framework. The CDR sequences may be situated, as an example, in the region of around amino acid residues 26-35; 53-59; and 98-117 for CDR1, CDR2 and CDR3, respectively, of the provided exemplary variable region sequences set forth in SEQ ID NOs: 16 to 50. It will be understood by one of skill in the art that the CDR sequences may be in different positions if a different framework sequence is selected, although generally the order of the sequences will remain the same.

The CDR1 and CDR2 sequences of the anti-BCMA antibodies of the present invention may be encompassed by the following structural formulas, where an X indicates a variable amino acid, which may be specific amino acids as indicated below.

CDR1
(SEQ ID NO: 20)
G F T F X1 X2 X3 A

where
X1 is S or T;
X2 is S or N;
X3 is H or Y.

In one embodiment, both X1 and X2 are S. In another embodiment, X3 is H. In a further embodiment, X1 X2 X3 has the sequence SSH. In other embodiments, CDR1 comprises the sequence GFTFSSHA (SEQ ID NO: 2) or the sequence GFTFSSYA (SEQ ID NO: 3) or the sequence of GFTFTNHA (SEQ ID NO: 1).

CDR2
(SEQ ID NO: 21)
I S G X4 G X5 X6 X7

where
X4 is S or N;
X5 is D or R;
X6 is T, F or Y; and
X7 is T or I.

In one embodiment, X4 is S. In another embodiment, X5 is D. In a further embodiment, X4 is S and X5 is D. In a still further embodiment, X6 is Y. In another embodiment, X4 is S, X5 is D and X6 is Y. In yet another embodiment, X7 is T. In a further embodiment, X4 is S, X5 is D and X7 is T. In other embodiments, CDR2 comprise the sequence of SEQ ID NO: 4, or SEQ ID NO: 5, or SEQ ID NO: 6, or SEQ ID NO: 7.

In one embodiment, CDR3 is selected from the group consisting of AKDGGETLVDS (SEQ ID NO: 8), AKDEDGGSLLGY (SEQ ID NO: 9), AKDEDGGSLLGH (SEQ ID NO: 10), and AKEGTGANSSLADY (SEQ ID NO: 11).

Representative CDR1, CDR2, and CDR3 sequences are shown in FIG. 1.

In one embodiment, the anti-BCMA heavy chain-only antibody of the present invention comprises the CDR1 sequence of SEQ ID NO: 1; the CDR2 sequence of SEQ ID NO: 4 and a CDR3 sequence of SEQ ID NO: 8.

In another embodiment, the anti-BCMA heavy chain-only antibody of the present invention comprises the CDR1 sequence of SEQ ID NO: 2; a CDR2 sequence of SEQ ID NO: 5; and a CDR3 sequence of SEQ ID NO: 9.

In a further embodiment, the anti-BCMA heavy chain-only antibody of the present invention comprises the CDR1 sequence of SEQ ID NO:2; the CDR2 sequence of SEQ ID NO: 6; and the CDR3 sequence of SEQ ID NO: 10.

In a still further embodiment, the anti-BCMA heavy chain-only antibody of the present invention comprises the CDR1 sequence of SEQ ID NO: 3, the CDR2 sequence of SEQ ID NO: 7, and the CDR3 sequence of SEQ ID NO: 11.

In further embodiments, the anti-BCMA antibody of the present invention comprises any of the heavy chain variable region amino acid sequences of SEQ ID NOs: 12 to 15 (FIG. 2).

In some embodiments, a CDR sequence in the anti-BMA antibodies of the present invention comprises two or fewer amino acid substitutions relative to a CDR1, CDR2 and/or CDR3 sequence or set of CDR1, CDR2 and CDR3 sequences in any one of SEQ ID NOs:1 to 11 (FIG. 1). In some embodiments, said amino acid substitution(s) are one or two of amino acid positions 5-7 of CDR1, and/or one or two of the amino acid positions 4, 6, 8 of CDR2, and/or one or two amino acid positions of CDR3, relative to the formulas and sequences provided above. In some embodiments, the heavy chain-only anti-BCMA antibodies herein will comprise a heavy chain variable region sequence with at least 85% identity, at least 90% identity, at least 95% identity, at least 98% identify, or at least 99% identity to any of the heavy chain variable region sequences shown in FIG. 2 (SEQ ID NOs: 12-15).

In some embodiments, bispecific or multispecific antibodies are provided, which may have any of the configurations discussed herein, including, without limitation, a three chain bispecific antibody. Bispecific antibodies comprise at least the heavy chain variable region of an antibody specific for a protein other than BCMA.

Where a protein of the invention is a bispecific antibody, one binding moiety is specific for human BCMA while the other arm may be specific for target cells, tumor associated antigens, targeting antigens, e.g., integrins, etc., pathogen antigens, checkpoint proteins, and the like. Target cells specifically include cancer cells, such as hematologic tumors, e.g., B-cell tumors, as discussed below.

Various formats of bispecific antibodies are within the ambit of the invention, including, without limitation, single chain polypeptides, two chain polypeptides, three chain polypeptides, four chain polypeptides, and multiples thereof. The bispecific antibodies herein specifically include T-cell bispecific antibodies binding to BCMA, which is selectively expressed on plasma cells (PCs) and multiple myeloma (MM), and CD3 (anti-BCMA×anti-CD3 antibodies). Such antibodies induce potent T-cell mediated killing of cells carrying BCMA, and can be used to treat tumors, in particular hematologic tumors, such as B-cell tumors, as discussed below.

Bispecific antibodies against CD3 and BCMA are described, for example, in WO2007117600, WO2009132058, WO2012066058, WO2012143498, WO2013072406, WO2013072415, and WO2014122144, and in US 20170051068.

### Pharmaceutical Compositions

It is another aspect of the present invention to provide pharmaceutical compositions comprising one or more antibodies of the present invention in admixture with a suitable pharmaceutically acceptable carrier. Pharmaceutically acceptable carriers as used herein are exemplified, but not limited to, adjuvants, solid carriers, water, buffers, or other carriers used in the art to hold therapeutic components, or combinations thereof.

Pharmaceutical composition of the antibodies used in accordance with the present invention are prepared for storage by mixing proteins having the desired degree of purity with optional pharmaceutically acceptable carriers, excipients or stabilizers (see, e.g., Remington's Pharmaceutical Sciences 16th edition, Osol, A. Ed. (1980)), such as in the form of lyophilized formulations or aqueous solutions. Acceptable carriers, excipients, or stabilizers are nontoxic to recipients at the dosages and concentrations employed, and include buffers such as phosphate, citrate, and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g., Zn-protein complexes); and/or non-ionic surfactants such as TWEEN™, PLURONICS™ or polyethylene glycol (PEG).

Pharmaceutical compositions for parenteral administration are preferably sterile and substantially isotonic and manufactured under Good Manufacturing Practice (GMP) conditions. Pharmaceutical compositions can be provided in unit dosage form (i.e., the dosage for a single administration). The formulation depends on the route of administration chosen. The antibodies herein can be administered by intravenous injection or infusion or subcutaneously. For injection administration, the antibodies herein can be formulated in aqueous solutions, preferably in physiologically-compatible buffers to reduce discomfort at the site of injection. The solution can contain carriers, excipients, or stabilizers as discussed above. Alternatively, antibodies can be in lyophilized form for constitution with a suitable vehicle, e.g., sterile pyrogen-free water, before use.

Anti-BCMA antibody formulations are disclosed, for example, in U.S. Pat. No. 9,034,324. Similar formulations can be used for the proteins of the present invention. Subcutaneous antibody formulations are described, for example, in US 20160355591 and US 20160166689.

### Methods of Use

The pharmaceutical compositions herein can be used for the treatment of B-cell related disorders, including B-cell and plasma cell malignancies and autoimmune disorders characterized by the expression or overexpression of BCMA.

Such B-cell related disorders include B-cell and plasma cell malignancies and autoimmune disorders, including, without limitation, plasmacytoma, Hodgkins' lymphoma, follicular lymphomas, small non-cleaved cell lymphomas, endemic Burkitt's lymphoma, sporadic Burkitt's lymphoma, marginal zone lymphoma, extranodal mucosa-associated lymphoid tissue lymphoma, nodal monocytoid B cell lymphoma, splenic lymphoma, mantle cell lymphoma, large cell lymphoma, diffuse mixed cell lymphoma, immunoblastic lymphoma, primary mediastinal B cell lymphoma, pulmonary B cell angiocentric lymphoma, small lymphocytic lymphoma, B cell proliferations of uncertain malignant potential, lymphomatoid granulomatosis, post-transplant lymphoproliferative disorder, an immunoregulatory disorder, rheumatoid arthritis, myasthenia gravis, idiopathic thrombocytopenia purpura, anti-phospholipid syndrome, Chagas' disease, Grave's disease, Wegener's granulomatosis, poly-arteritis nodosa, Sjogren's syndrome, pemphigus vulgaris, scleroderma, multiple sclerosis, anti-phospholipid syndrome, ANCA associated vasculitis, Goodpasture's disease, Kawasaki disease, autoimmune hemolytic anemia, and rapidly progressive glomerulonephritis, heavy-chain disease, primary or immunocyte-associated amyloidosis, or monoclonal gammopathy.

The plasma cell disorders characterized by the expression of BCMA include Multiple Myeloma (MM). MM is a B-cell malignancy characterized by a monoclonal expansion and accumulation of abnormal plasma cells in the bone marrow compartment. Current therapies for MM often cause remissions, but nearly all patients eventually relapse and die. There is substantial evidence of an immune-mediated elimination of myeloma cells in the setting of allogeneic hematopoietic stem cell transplantation; however, the toxicity of this approach is high, and few patients are cured. Although some monoclonal antibodies have shown promise for treating MM in preclinical studies and early clinical trials, consistent clinical efficacy of any monoclonal antibody therapy for MM has not been conclusively demonstrated. There is therefore a great need for new therapies, including immunotherapies, for MM (see, e.g. Carpenter et al., Clin Cancer Res 2013, 19(8):2048-2060).

Overexpression or activation of BCMA by its proliferation-inducing ligand, APRIL it known to promote human Multiple Myeloma (MM) progression in vivo. BCMA has also been shown to promote in vivo growth of xenografted MM cells harboring p53 mutation in mice. Since activity of the APRIL/BCMA pathway plays a central role in MM pathogenesis and drug resistance via bidirectional interactions between tumor cells and their supporting bone marrow microenvironment, BCMA has been identified as a target for the treatment of MM. For further details see, e.g., Yu-Tsu Tai et al., Blood 2016; 127(25):3225-3236.

Another B-cell disorder involving plasma cells expressing BCMA is systemic lupus erythematosus (SLE), also known as lupus. SLE is a systemic, autoimmune disease that can affect any part of the body and is represented with the immune system attacking the body's own cells and tissue, resulting in chronic inflammation and tissue damage. It is a Type III hypersensitivity reaction in which antibody-immune complexes precipitate and cause a further immune response (Inaki & Lee, Nat Rev Rheumatol 2010; 6: 326-337).

The anti-BCMA heavy chain-only antibodies (UniAbs) of the present invention can be used to develop therapeutic agents for the treatment of MM, SLE, and other B-cell disorders or plasma cell disorders characterized by the expression of BCMA, such as those listed above. In particular, the anti-BCMA heavy chain-only antibodies (UniAbs) of the present invention are candidates for the treatment of MM, alone or in combination with other MM treatments.

In one embodiment, the antibodies herein can be in the form of heavy chain-only anti-BCMA antibody-CAR structures, i.e., heavy chain-only anti-BCMA antibody-CAR-transduced T cell structures.

Effective doses of the compositions of the present invention for the treatment of disease vary depending upon many different factors, including means of administration, target site, physiological state of the patient, whether the patient is human or an animal, other medications administered, and whether treatment is prophylactic or therapeutic. Usually, the patient is a human, but nonhuman mammals may also be treated, e.g., companion animals such as dogs, cats, horses, etc., laboratory mammals such as rabbits, mice, rats, etc., and the like. Treatment dosages can be titrated to optimize safety and efficacy.

Dosage levels can be readily determined by the ordinarily skilled clinician, and can be modified as required, e.g., as required to modify a subject's response to therapy. The amount of active ingredient that can be combined with the carrier materials to produce a single dosage form varies depending upon the host treated and the particular mode of administration. Dosage unit forms generally contain between from about 1 mg to about 500 mg of an active ingredient.

In some embodiments, the therapeutic dosage of the agent may range from about 0.0001 to 100 mg/kg, and more usually 0.01 to 5 mg/kg, of the host body weight. For example, dosages can be 1 mg/kg body weight or 10 mg/kg body weight or within the range of 1-10 mg/kg. An exemplary treatment regimen entails administration once every two weeks or once a month or once every 3 to 6 months. Therapeutic entities of the present invention are usually administered on multiple occasions. Intervals between single dosages can be weekly, monthly or yearly. Intervals can also be irregular as indicated by measuring blood levels of the therapeutic entity in the patient. Alternatively, therapeutic entities of the present invention can be administered as a sustained release formulation, in which case less frequent administration is required. Dosage and frequency vary depending on the half-life of the polypeptide in the patient.

Typically, compositions are prepared as injectables, either as liquid solutions or suspensions; solid forms suitable for solution in, or suspension in, liquid vehicles prior to injection can also be prepared. The pharmaceutical compositions herein are suitable for intravenous or subcutaneous administration, directly or after reconstitution of solid (e.g., lyophilized) compositions. The preparation also can be emulsified or encapsulated in liposomes or micro particles such as polylactide, polyglycolide, or copolymer for enhanced adjuvant effect, as discussed above. Langer, Science 249: 1527, 1990 and Hanes, Advanced Drug Delivery Reviews 28: 97-119, 1997. The agents of this invention can be administered in the form of a depot injection or implant preparation which can be formulated in such a manner as to permit a sustained or pulsatile release of the active ingredient. The pharmaceutical compositions are generally formulated as sterile, substantially isotonic and in full compliance with all Good Manufacturing Practice (GMP) regulations of the U.S. Food and Drug Administration.

Toxicity of the antibodies and antibody structures described herein can be determined by standard pharmaceutical procedures in cell cultures or experimental animals, e.g., by determining the LD50 (the dose lethal to 50% of the population) or the LD100 (the dose lethal to 100% of the population). The dose ratio between toxic and therapeutic effect is the therapeutic index. The data obtained from these cell culture assays and animal studies can be used in formulating a dosage range that is not toxic for use in humans. The dosage of the antibodies described herein lies preferably within a range of circulating concentrations that include the effective dose with little or no toxicity. The dosage can vary within this range depending upon the dosage form employed and the route of administration utilized. The exact formulation, route of administration and dosage can be chosen by the individual physician in view of the patient's condition.

The compositions for administration will commonly comprise an antibody or other ablative agent dissolved in a pharmaceutically acceptable carrier, preferably an aqueous carrier. A variety of aqueous carriers can be used, e.g., buffered saline and the like. These solutions are sterile and generally free of undesirable matter. These compositions may be sterilized by conventional, well known sterilization techniques. The compositions may contain pharmaceutically acceptable auxiliary substances as required to approximate physiological conditions such as pH adjusting and buffering agents, toxicity adjusting agents and the like, e.g., sodium acetate, sodium chloride, potassium chloride, calcium chloride, sodium lactate and the like. The concentration of active agent in these formulations can vary widely, and will be selected primarily based on fluid volumes, viscosities, body weight and the like in accordance with the particular mode of administration selected and the patient's needs (e.g., Remington's Pharmaceutical Science (15th ed., 1980) and Goodman & Gillman, The Pharmacological Basis of Therapeutics (Hardman et al., eds., 1996)).

Also within the scope of the invention are kits comprising the active agents of the invention, and formulations thereof, and instructions for use. The kits can further contain a least one additional reagent, e.g., a chemotherapeutic drug, etc. Kits typically include a label indicating the intended use of the contents of the kit. The term label includes any writing, or recorded material supplied on or with the kit, or which otherwise accompanies the kit.

The invention now being fully described, it will be apparent to one of ordinary skill in the art that various changes and modifications can be made without departing from the spirit or scope of the invention.

### EXAMPLES

### Example 1

### Genetically Engineered Rats Expressing Heavy Chain-Only Antibodies

A ‘human—rat’ IgH locus was constructed and assembled in several parts. This involved the modification and joining of rat C region genes downstream of human JHs and subsequently, the upstream addition of the human VH6-D-segment region. Two BACs with separate clusters of human VH genes [BAC6 and BAC3] were then co-injected with the BAC termed Georg, encoding the assembled and modified region comprising human VH6, all Ds, all JHs, and modified rat Cγ2a/1/2b (ΔCH1).

Transgenic rats carrying artificial heavy chain immunoglobulin loci in unrearranged configuration were generated. The IgG2a(ΔC_{H}1)., igG1(ΔC_{H}1)., IgG2b(ΔC_{H}1) genes lacked the C_{H}1 segment. The constant region genes IgE, IgA and 3′ enhancer were included in Georg BAC. RT-PCR and serum analysis (ELISA) of transgenic rats revealed productive rearrangement of transgenic immunoglobulin loci and expression of heavy chain only antibodies of various isotypes in serum. Transgenic rats were cross-bred with rats with mutated endogenous heavy chain and light chain loci previously described in US patent publication 2009/0098134 A1. Analysis of such animals demonstrated inactivation of rat immunoglobulin heavy and light chain expression and high level expression of heavy chain antibodies with variable regions encoded by human V, D, and J genes. Immunization of transgenic rats resulted in production of high titer serum responses of antigen-specific heavy chain antibodies. These transgenic rats expressing heavy chain antibodies with a human VDJ region were called UniRats.

### Example 2

### Immunization

### Immunization with Recombinant Extracellular Domain of BCMA

Twelve UniRat animals (6 HC27, 6 HC28) were immunized with recombinant human BCMA protein. The animals were immunized according to standard protocol using a Titermax/Alhydrogel adjuvant. Recombinant extracellular domain of BCMA was purchased from R&D Systems and was diluted with sterile saline and combined with adjuvant. The immunogen was combined with Titermax and Alhydrogel adjuvants. The first immunization (priming) with immunogen in Titermax was administered in the left and right legs. Subsequent boosting immunizations were done in the presence of Alhydrogel and three days before harvest boosts were performed with immunogens in PBS. Serum was collected from rats at the final bleed to determine serum titers.

### Serum Titer Results

Binding activity for a single 1:500 serum titer dilution is tested by ELISA against a huBCMA+Fc protein and a cynoBCMA+Fc protein produced in eukaryotic cells and two human BCMA proteins from E. coli and wheat germ, respectively. In addition, serum samples are tested against two off-target proteins, HSA and human IgG1. In addition, serum from all animals is assayed for binding to NCI-H929 cells (BCMA+, lambda−).

Since usually a significant spread of results is observed in serum reactivity levels to NCI-H929 cells (BCMA+, lambda−), the relevance of these results is confirmed by the ELISA binding data generated for a subset of the animals. Positive signal for binding to the cynoBCMA+Fc protein may reflect binding to either the ECD or the Fc portion of the molecule that is also included on the human immunogen. In both assay types, analysis of serum taken from these animals prior to immunization showed no reactivity to the immunogen or off target protein.

### Example 3

### Gene Assembly, Expression and Binding Assays

cDNAs encoding heavy chain only antibodies highly expressed in lymph node cells were selected for gene assembly and cloned into an expression vector. Subsequently, these heavy chain sequences were expressed in HEK cells as UniAb heavy chain only antibodies (CH1 deleted, no light chain).

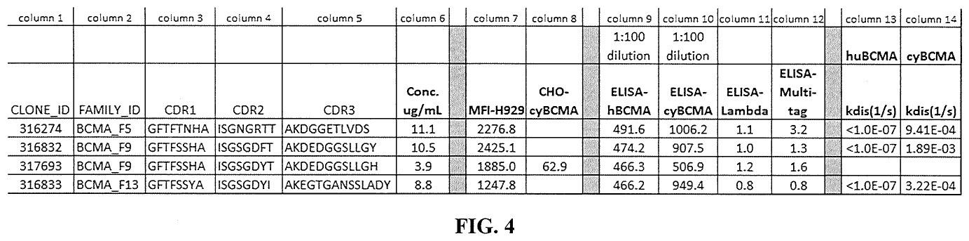

The results of assays testing the binding of the anti-BCMA heavy chain-only antibodies of the invention to BCMA protein (human and cynomolgus) and a BCMA-expressing cell line (H929; BCMA+, lambda−) at various concentrations are shown in FIG. 4. The NCI-H929 cell line is human multiple myeloma line expressing human BCMA, which was obtained from the American Type Culture Collection (ATCC) and cultured according to ATCC recommendations.

Supernatants of 4 antibodies were tested for binding in a standard ELISA assay to human and cynomolgus BCMA. Binding to recombinant BCMA protein was determined by ELISA using human BCMA ECD obtained from Abcam (ab50089). The BCMA ECD protein was used at a concentration of 2 μg/mL to capture UniAbs at 50 ng/mL. Binding of UniAbs was detected with a goat anti-human IgG HRP conjugated antibody (ThermoFisher 31413). All antibodies were diluted in 1X TBS with 0.05% Tween-20 and 1% dry milk powder.

Off-target binding of human IgG1 was assessed by ELISA using the UniAbs to capture human IgG1 kappa followed by detection of the kappa chain with a goat anti-human kappa HRP conjugated antibody (Southern Biotech 2060-05).

Supernatants of the 4 test anti-BCMA antibodies were also tested by flow cytometry for binding to H929 cells. The samples were measured by flow cytometry using a Guava easyCyte 8HT instrument from EMD Millipore and analyzed using guavaSoft. Bound antibodies were detected with goat anti-human IgG F(ab)2 conjugated to PE (Southern Biotech 2042-09). All antibodies were diluted in PBS with 1% BSA. Positive staining was determined by comparison to staining with a human IgG1 isotype control.

In FIG. 4: Column 1 indicates the clone ID of the HCAb. Column 2 indicates the family ID of the HCAb based on the CDR3 sequence. Column 3 indicates the CDR1 amino acid sequence. Column 4 indicates the CDR2 amino acid sequence. Column 5 indicates the CDR3 amino acid sequence. Column 6 indicates the concentration of the expressed HCAb in ug/mL. Column 7 indicates the mean fluorescent intensity of cell binding to H929 human cells that express BCMA. Column 8 indicates the mean fluorescent intensity of cell binding to CHO cells that express cyno BCMA. Column 9 indicates the ELISA fold over background signal of human BCMA protein binding. Column 10 indicates the ELISA fold over background signal of cyno BCMA protein binding. Column 11 indicates the ELISA fold over background signal of lambda protein binding, an off-target control. Column 12 indicates the ELISA fold over background signal of a multi-tag protein binding, an off-target control. Column 13 indicates the binding off-rate to human BCMA protein measured by the Octet. Column 14 indicates the binding off-rate to cyno BCMA protein measured by the Octet.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

1.-29. (canceled)

30. A heavy chain-only antibody binding to human B-Cell Maturation Antigen (BCMA) comprising a heavy chain variable region comprising:
(a) a CDR1 sequence of the formula
G F T F X1 X2X3 A
where
X1 is S or T;
X2 is S or N;
X3 is H or Y; and
(b) a CDR2 sequence of the formula
I S G X4G X5X6X7
where
X4 is S or N;
X5 is D or R;
X6 is T, F or Y;
X7 is T or I; and
(c) a CDR3 sequence selected from the group consisting of AKDGGETLVDS (SEQ ID NO: 8), AKDEDGGSLLGY (SEQ ID NO: 9), AKDEDGGSLLGH (SEQ ID NO: 10), and AKEGTGANSSLADY (SEQ ID NO: 11).

31. The heavy chain-only antibody of claim 30, wherein the CDR1, CDR2, and CDR3 sequences are present in a human framework.

32. The heavy chain-only antibody of claim 30, further comprising a heavy chain constant region sequence in the absence of a CH1 sequence.

33. The heavy chain-only antibody of claim 30, comprising a heavy chain variable region having at least 95% sequence identity to SEQ ID NO: 12.

34. The heavy chain-only antibody of claim 33, comprising a heavy chain variable region sequence comprising SEQ ID NO: 12.

35. The heavy chain-only antibody of claim 30, comprising a heavy chain variable region having at least 95% sequence identity to SEQ ID NO: 13.

36. The heavy chain-only antibody of claim 35, comprising a heavy chain variable region sequence comprising SEQ ID NO: 13.

37. The heavy chain-only antibody of claim 30, comprising a heavy chain variable region having at least 95% sequence identity to SEQ ID NO: 14.

38. The heavy chain-only antibody of claim 37, comprising a heavy chain variable region sequence comprising SEQ ID NO: 14.

39. The heavy chain-only antibody of claim 30, which is multi-specific.

40. The heavy chain-only antibody of claim 39, which is bispecific.

41. The heavy chain-only antibody of claim 39, having binding affinity to an effector cell.

42. The heavy chain-only antibody of claim 39, having binding affinity to a T-cell antigen.

43. The heavy chain-only antibody of claim 42, having binding affinity to CD3.

44. The heavy chain-only antibody of claim 30, which is in a CAR-T format.

45. The heavy chain-only antibody of claim 44, which is present in a CAR-transduced T-cell.

46. A T-cell transduced with the heavy chain-only antibody of claim 44.

47. A pharmaceutical formulation comprising a heavy chain-only antibody and a pharmaceutically acceptable excipient, wherein the heavy chain-only antibody binds human BCMA and comprise a heavy chain variable region comprising:
(a) a CDR1 sequence of the formula
G F T F X1 X2X3 A
where
X1 is S or T;
X2 is S or N;
X3 is H or Y; and
(b) a CDR2 sequence of the formula
I S G X4G X5X6X7
where
X4 is S or N;
X5 is D or R;
X6 is T, F or Y;
X7 is T or I; and
(c) a CDR3 sequence selected from the group consisting of AKDGGETLVDS (SEQ ID NO: 8), AKDEDGGSLLGY (SEQ ID NO: 9), AKDEDGGSLLGH (SEQ ID NO: 10), and AKEGTGANSSLADY (SEQ ID NO: 11).

48. A method for the treatment of a B-cell disorder characterized by the expression of BCMA, comprising administering to a subject with said disorder a heavy chain-only antibody binding to human BCMA comprising a heavy chain variable region comprising:
(a) a CDR1 sequence of the formula
G F T F X1 X2X3 A
where
X1 is S or T;
X2 is S or N;
X3 is H or Y; and
(b) a CDR2 sequence of the formula
I S G X4G X5X6X7
where
X4 is S or N;
X5 is D or R;
X6 is T, F or Y;
X7 is T or I; and
(c) a CDR3 sequence selected from the group consisting of AKDGGETLVDS (SEQ ID NO: 8), AKDEDGGSLLGY (SEQ ID NO: 9), AKDEDGGSLLGH (SEQ ID NO: 10), and AKEGTGANSSLADY (SEQ ID NO: 11).

49. The method of claim 48, wherein the B-cell disorder is multiple myeloma.

50. The method of claim 48, wherein the B-cell disorder is systemic lupus erythematosus.


SEQUENCE LISTING (21 sequences)

SEQ ID NO: 1 - protein, 8 aa, synthetic construct
  REGION 1..8: note = source = /note="Description of Artificial Sequence: Synthetic peptide"
GFTFTNHA

SEQ ID NO: 2 - protein, 8 aa, synthetic construct
  REGION 1..8: note = source = /note="Description of Artificial Sequence: Synthetic peptide"
GFTFSSHA

SEQ ID NO: 3 - protein, 8 aa, synthetic construct
  REGION 1..8: note = source = /note="Description of Artificial Sequence: Synthetic peptide"
GFTFSSYA

SEQ ID NO: 4 - protein, 8 aa, synthetic construct
  REGION 1..8: note = source = /note="Description of Artificial Sequence: Synthetic peptide"
ISGNGRTT

SEQ ID NO: 5 - protein, 8 aa, synthetic construct
  REGION 1..8: note = source = /note="Description of Artificial Sequence: Synthetic peptide"
ISGSGDFT

SEQ ID NO: 6 - protein, 8 aa, synthetic construct
  REGION 1..8: note = source = /note="Description of Artificial Sequence: Synthetic peptide"
ISGSGDYT

SEQ ID NO: 7 - protein, 8 aa, synthetic construct
  REGION 1..8: note = source = /note="Description of Artificial Sequence: Synthetic peptide"
ISGSGDYI

SEQ ID NO: 8 - protein, 11 aa, synthetic construct
  REGION 1..11: note = source = /note="Description of Artificial Sequence: Synthetic peptide"
AKDGGETLVDS

SEQ ID NO: 9 - protein, 12 aa, synthetic construct
  REGION 1..12: note = source = /note="Description of Artificial Sequence: Synthetic peptide"
AKDEDGGSLLGY

SEQ ID NO: 10 - protein, 12 aa, synthetic construct
  REGION 1..12: note = source = /note="Description of Artificial Sequence: Synthetic peptide"
AKDEDGGSLLGH

SEQ ID NO: 11 - protein, 14 aa, synthetic construct
  REGION 1..14: note = source = /note="Description of Artificial Sequence: Synthetic peptide"
AKEGTGANSSLADY

SEQ ID NO: 12 - protein, 118 aa, synthetic construct
  REGION 1..118: note = source = /note="Description of Artificial Sequence: Synthetic polypeptide"
QVQLVESGGGLVQPGGSLRLSCAASGFTFTNHAMSWVRQAPGKGLELVSSISGNGRTTYYADSVKGRFTISRDISKNTLDLQMNSLRAEDTAVYYCAKDGGETLVDSRGQGTLVTVSS

SEQ ID NO: 13 - protein, 119 aa, synthetic construct
  REGION 1..119: note = source = /note="Description of Artificial Sequence: Synthetic polypeptide"
QVQLVESGGGLVQPGGSLRLSCAASGFTFSSHAMTWVRQAPGKGLEWVAAISGSGDFTHYADSVKGRFTISRDNSKNTVSLQMNNLRAEDTAVYYCAKDEDGGSLLGYRGQGTLVTVSS

SEQ ID NO: 14 - protein, 119 aa, synthetic construct
  REGION 1..119: note = source = /note="Description of Artificial Sequence: Synthetic polypeptide"
EVQLLESGGGLIQPGGSLRLSCAASGFTFSSHAMTWVRQAPGKGLEWVSAISGSGDYTHYADSVKGRFTISRDNSKNTVYLQMNSLRAEDSAVYYCAKDEDGGSLLGHRGQGTLVTVSS

SEQ ID NO: 15 - protein, 121 aa, synthetic construct
  REGION 1..121: note = source = /note="Description of Artificial Sequence: Synthetic polypeptide"
EVQLLESGGGLVQPGGSLRLSCAASGFTFSSYAMSWVRQAPGKGLEWVSSISGSGDYIYYADSVKGRFTISRDISKNTLYLQMNSLRAEDTAVYYCAKEGTGANSSLADYRGQGTLVTVSS

SEQ ID NO: 16 - DNA, 354 bp, synthetic construct
  misc_feature 1..354: note = source = /note="Description of Artificial Sequence: Synthetic polynucleotide"
  source 1..354: mol_type = other DNA
caggtgcagctggtggagtctgggggaggcttggtacagcctggggggtccctgagactctcctgtgcagcctctggattcacctttaccaaccatgccatgagttgggtccgccaggctccagggaaggggctggagttggtctcaagtattagtggtaatggtcgtaccacatactacgcagactccgtgaagggccggttcaccatctccagagacatttccaagaacacgctggatctgcaaatgaacagcctgagagccgaggacacggccgtatattactgtgcgaaagatgggggcgaaactctagttgactccagaggccagggcaccctggtcaccgtctcctca

SEQ ID NO: 17 - DNA, 357 bp, synthetic construct
  misc_feature 1..357: note = source = /note="Description of Artificial Sequence: Synthetic polynucleotide"
  source 1..357: mol_type = other DNA
caggtgcagctggtggagtctgggggaggcttggtacagcctggggggtccctgagactctcctgtgcagcctctggattcacctttagcagccatgccatgacctgggtccgccaggctccggggaaggggctggagtgggtcgcagctattagtggcagtggtgatttcacacactacgcagactccgtgaagggccggttcaccatctccagagacaattccaagaacacggtgtctctgcaaatgaacaacctgagagccgaggacacggccgtatattactgtgcgaaagatgaggatggtgggagcttgcttggctacagaggccagggcaccctggtcaccgtctcctca

SEQ ID NO: 18 - DNA, 357 bp, synthetic construct
  misc_feature 1..357: note = source = /note="Description of Artificial Sequence: Synthetic polynucleotide"
  source 1..357: mol_type = other DNA
gaggtgcagctgttggagtctggggggggcttgatacagcctggggggtccctgagactctcctgtgcagcctctggattcacctttagcagccatgccatgacctgggtccgccaggctccggggaaggggctggagtgggtctcagctattagtggtagtggtgattacacacactacgcagactccgtgaagggtcggttcaccatctccagagacaattccaagaacacggtgtatctccaaatgaacagtctgagagccgaggactcggccgtatattactgtgcgaaagatgaggatggtgggagcctcctggggcacagaggccagggcaccctggtcaccgtctcctca

SEQ ID NO: 19 - DNA, 363 bp, synthetic construct
  misc_feature 1..363: note = source = /note="Description of Artificial Sequence: Synthetic polynucleotide"
  source 1..363: mol_type = other DNA
gaggtgcagctgttggagtctgggggaggcttggtacagcctggggggtccctgagactctcctgtgcagcctctggattcacctttagcagctatgccatgagctgggtccgccaggctccagggaaggggctggagtgggtctcatctattagtggtagtggtgattacatatactacgcagactccgtgaagggccggttcaccatctccagagacatatccaagaacacgctgtatctgcaaatgaacagcctgagagccgaggacacggccgtatattactgtgcgaaagaaggtacgggtgccaacagcagcttggcagactacagaggccagggcaccctggtcaccgtctcctca

SEQ ID NO: 20 - protein, 8 aa, synthetic construct
  VARIANT 5: note = /replace="Thr"
  VARIANT 6: note = /replace="Asn"
  VARIANT 7: note = /replace="Tyr"
  SITE 1..8: note = /note="Variant residues given in the sequence have no preference with respect to those in the annotations for variant positions"
  REGION 1..8: note = source = /note="Description of Artificial Sequence: Synthetic peptide"
GFTFSSHA

SEQ ID NO: 21 - protein, 8 aa, synthetic construct
  VARIANT 4: note = /replace="Asn"
  VARIANT 6: note = /replace="Arg"
  VARIANT 7: note = /replace="Phe" or "Tyr"
  VARIANT 8: note = /replace="Ile"
  SITE 1..8: note = /note="Variant residues given in the sequence have no preference with respect to those in the annotations for variant positions"
  REGION 1..8: note = source = /note="Description of Artificial Sequence: Synthetic peptide"
ISGSGDTT